(19) United States
(12) Patent Application Publication
(10) Pub. No.: US 2017/0196577 A1
(43) Pub. Date: Jul. 13, 2017

(54) GUIDEWIRES, SYSTEMS AND METHODS OF USE

(71) Applicant: BOSTON SCIENTIFIC SCIMED, INC., Maple Grove, MN (US)

(72) Inventors: Erik E. Sperry, Newburyport, MA (US); Jerry T. Long, Jr., Jamaica Plain, MA (US); Ronald Ciulla, Westford, MA (US); Aaron K. Kirkemo, Gladstone, NJ (US); Kenneth P. Reever, Hopedale, MA (US); Brandon W. Craft, Phoenix, MD (US); Elizabeth A. Stokley, Baltimore, MD (US); Anant Subramaniam, Owings Mills, MD (US)

(21) Appl. No.: 15/398,938

(22) Filed: Jan. 5, 2017

Related U.S. Application Data
(60) Provisional application No. 62/275,860, filed on Jan. 7, 2016.

(51) Int. Cl. A61B 17/221; A61M 29/02

(52) U.S. Cl. CPC A61B 17/221; A61M 29/02; A61B 2017/22042

(57) ABSTRACT

A guidewire including an elongate inner member having a proximal portion and a distal portion, an elongate outer member disposed about the elongate inner member, wherein one of the elongate inner member and the elongate outer member is more flexible relative to the other of the elongate inner member or the elongate outer member, and the elongate inner member is movable relative to the elongate outer member.

### CROSS-REFERENCE TO RELATED APPLICATIONS

This application claims priority to U.S. Provisional Patent Application Ser. No. 62/275,860, filed on Jan. 7, 2016, the contents of which are fully incorporated herein by reference.

### FIELD

The disclosure generally pertains to intravascular guidewires.

### BACKGROUND

Minimally invasive medical procedures or percutaneous medical procedures are performed through tiny incisions in the skin and are preferentially used where possible due to quicker patient recovery times and less discomfort. Various medical devices are used to provide access to remote surgical sites within a human body through body openings, cavities, tracts, blood vessels, arteries and so forth. Such surgical devices may include elongate portions that are maneuverable in the openings and that allow an operator to simultaneously view and operate at the remote site.

A wide variety of guidewires have been developed for use in these minimally invasive procedures. Guidewires are commonly used in conjunction with medical devices such as catheters, needles, scopes, retrieval devices, and so forth, to facilitate navigation through the body of a patient. Because these routes may be tortuous or require manipulation around foreign or biological materials in the patient, it is desirable to combine a number of performance features in an guidewire. For example, it is sometimes desirable that the guidewire have a relatively high level of pushability and torqueability, particularly near its proximal end. It is also sometimes desirable that a guidewire be relatively flexible particularly near its distal end. A number of different guidewire structures and assemblies are known, each having certain advantages and disadvantages. However, there is an ongoing need to provide alternative guidewire structures and assemblies.

### SUMMARY

This disclosure provides design, material, manufacturing method, and use alternatives for medical devices. In one aspect, the disclosure relates to a guidewire including an elongate inner member having a proximal portion and a distal portion and an elongate outer member disposed about the elongate inner member. One of the elongate inner member and the elongate outer member is more flexible relative to the other of the elongate inner member or the elongate outer member. The elongate inner member is movable relative to the elongate outer member.

Alternatively or additionally, to any of the embodiments above, to any of the embodiments above, the elongate outer member is movable with respect to the elongate inner member, the elongate outer member can be pulled in a proximal direction relative to the elongate inner member, to shift the distal portion of the elongate inner member into an alternative shape.

Alternatively or additionally, to any of the embodiments above, to any of the embodiments above, the elongate inner member is movable with respect to the elongate outer member, the elongate inner member can be pulled in a proximal direction relative to the elongate outer member, to shift the distal portion of the elongate outer member into an alternative shape.

Alternatively or additionally, to any of the embodiments above, the distal portion of the elongate inner member is shiftable into an expanded state, in the expanded state, the distal portion of the elongate inner member comprises a geometry configured to anchor the guidewire with respect to its surroundings.

Alternatively or additionally, to any of the embodiments above, the distal portion of the elongate inner member is shifted into the expanded state by exposure to an increase in temperature.

Alternatively or additionally, to any of the embodiments above, the distal portion of the elongate inner is shifted into an expanded state by inflating the distal portion with an inflation fluid.

Alternatively or additionally, to any of the embodiments above, wherein the distal portion of the elongate inner member is shifted into an expanded state by exposure to an electric field.

Alternatively or additionally, to any of the embodiments above, at least the distal portion of the elongate inner member comprises a polymer material, a shape memory material, an electroactive polymer material or combinations thereof.

Alternatively or additionally, to any of the embodiments above, the elongate inner member comprises a shape memory material, the shape member material is a nickel-titanium alloy.

Alternatively or additionally, to any of the embodiments above, the geometry of the distal portion of the elongate inner member in the expanded state is cylindrical, helical, elliptical, umbrella shaped, tubular, or a combination thereof.

Alternatively or additionally, to any of the embodiments above, the distal portion of the elongate outer member is shiftable into an expanded state providing a geometry configured to position the guidewire with respect to its surroundings, the distal portion of the elongate outer member is shifted into the expanded state by an increase in temperature, by inflating with an inflation fluid or by exposure to an electric field.

Alternatively or additionally, to any of the embodiments above, the elongate inner member has a distal tip, the distal tip has a shape including flat, spatula shaped, spoon shaped, cylindrical, elliptical, curved, segmented with interlocking pieces, or a combination thereof.

Alternatively or additionally, to any of the embodiments above, the elongate inner member comprises stainless steel, nickel-chromium alloy, cobalt alloy, nickel-titanium alloy, or combinations thereof.

Alternatively of additionally, at least the distal portion of the elongate inner member comprises a shapeable material.

Alternatively or additionally, to any of the embodiments above, the guidewire may be a hollow member having a distal portion having a plurality of holes therein for perfusion of a contrast solution therethrough.

In another aspect, the disclosure relates to a guidewire having a shaft, the shaft having a distal tip that is flat, spatula shaped, spoon shaped, cylindrical, elliptical, curved, or segmented with interlocking pieces, the distal tip is configured to navigate past an occlusion in a body lumen.

In another aspect, the disclosure relates to a method of retrieving an object from a body organ percutaneously. The method includes inserting a needle into the body organ and advancing a guidewire through the needle into the body organ to position adjacent the object. The guidewire includes an elongate inner member and an elongate outer member disposed about an outer surface of the elongate inner member. One of the elongate inner member or elongate outer member is more flexible that the other of the elongate inner member and the elongate outer member. The elongate outer member is movable with respect to the elongate inner member, pulling back the elongate outer member or the elongate inner member to release a distal portion elongate outer member or the elongate inner member. A catheter may then be advanced over the guidewire. If the catheter includes an expandable medical balloon, the catheter is advanced over the guidewire until the distal tip of the balloon is in the organ, and the balloon is then expanded. The catheter can be preloaded with a working sheath and removal of the object from the body organ is performed through the sheath.

Alternatively or additionally, to any of the embodiments above, to any of the embodiments above, one of the elongate outer member or the elongate inner member has a distal tip expandable to an anchor shape, wherein pulling back one of the elongate outer member or the elongate inner member anchors the guidewire in the organ.

Alternatively or additionally, to any of the embodiments above, to any of the embodiments above, the anchor shape comprises a geometry including cylindrical, helical, elliptical, umbrella shaped, tubular, or a combination thereof.

Alternatively or additionally, to any of the embodiments above, to any of the embodiments above, the elongate inner member has a distal tip that is configured for maneuverability past an occlusion in a body lumen, the distal tip is flat, spatula shaped, spoon shaped, cylindrical, elliptical, curved, segmented with interlocking pieces, or a combination thereof.

Alternatively or additionally, to any of the embodiments above, to any of the embodiments above, the method is percutaneous nephrolithotomy, the body organ is a kidney, and the object is a kidney stone.

Alternatively or additionally, to any of the embodiments above, the sheath has a proximal portion that includes an anchoring member for retention of the proximal portion of the inner elongate member or the proximal portion of the elongate outer member.

Alternatively or additionally, to any of the embodiments above, the anchoring member includes wings having a slot for insertion of the proximal end of the guidewire therein.

Alternatively or additionally, to any of the embodiments above, the wings may include an expandable balloon for further retaining the guidewire in the slot between the balloon in an expanded state, and an inner surface of the slot.

### BRIEF DESCRIPTION OF THE DRAWINGS

FIG. 1 is a schematic overview of a kidney; to FIG. 2 illustrates a method for accessing the renal pelvis of a kidney;

FIG. 3 is a cross-sectional view of a dual core guidewire;

FIG. 4 is a cross-sectional view of a distal portion of an alternative embodiment of a guidewire;

FIG. 5 is a cross-sectional view of a distal portion of an alternative embodiment of a guidewire;

FIG. 6 is a side view of a distal portion of an alternative embodiment of a guidewire;

FIG. 7 is a side view of a distal portion of an alternative embodiment of a guidewire;

FIG. 8 is a cross-sectional view of a distal portion of an alternative embodiment of a guidewire;

FIG. 9 is a partial end view of a distal portion of an alternative embodiment of a guidewire;

FIGS. 10A-10C are side views of a distal portion of an alternative embodiment of a guidewire;

FIG. 11 is a side view of a distal portion of an alternative embodiment of a guidewire;

FIG. 12 is cross-sectional view through a portion of an example guidewire taken at section A-A in FIG. 11;

FIG. 13 is cross-sectional view through a portion of an example guidewire taken at section A-A in FIG. 11;

FIG. 14 is cross-sectional view through a portion of an example guidewire taken at section B-B in FIG. 12.

FIG. 15A is an end view of a distal portion of an example embodiment of a guidewire;

FIG. 15B is side view of an alternative embodiment of a guidewire;

FIG. 16A is a partial cross-sectional view of an example embodiment of a guidewire;

FIG. 16B is a partial cross-sectional view of an example embodiment of a guidewire;

FIG. 17A is a side view of a distal portion of an example embodiment of a guidewire in an unexpanded state;

FIG. 17B is a side view of a distal portion of a guidewire similar to that shown in FIG. 17A in an expanded state;

FIG. 18A is a side view of a distal portion of an example embodiment guidewire;

FIG. 18B is a partial cross-sectional view of a distal portion of a guidewire similar to that shown in FIG. 18A with tension applied;

FIG. 19A is a side view of a distal portion of an example embodiment of a guidewire with tension applied;

FIG. 19B is a side view of a distal portion of a guidewire similar to that shown in FIG. 19A when pushed in a distal direction;

FIG. 20A is a side view of a distal portion of an example embodiment of a guidewire wherein the distal portion is shiftable into an alternative shape;

FIG. 20B is a side view of a distal portion of a guidewire similar to that shown in FIG. 20A that has been shifted into an alternative shape;

FIGS. 21A-21C are side views a distal portion of an example embodiment of a guidewire wherein the distal portion is shiftable into an alternative shape;

FIG. 22 is a side view of a distal portion of an alternative embodiment of a guidewire;

FIG. 23A is an end view of the proximal end of a working sheath having a guidewire disposed therein;

FIG. 23B is an end view of the proximal end of a working sheath similar to that shown in FIG. 23A wherein the proximal end of the guidewire has been anchored in the proximal end of the sheath;

FIG. 23C is an end view of an alternative embodiment of the proximal end of a working sheath having a guidewire disposed therein.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The disclosure provides design, material, manufacturing method, and use alternatives for medical devices such as guidewires, catheters, catheter systems, endoscopic instruments, percutaneous nephrolithotmy (PCNL) catheters and catheter systems, and the like. Medical devices including devices and systems for endoscopic interventions that may access organs, such as the renal pelvis of the kidney are disclosed as well as methods for making and using such devices. An example method for accessing a body organ may include providing a needle having a lumen, a guidewire, a catheter system, and so forth. The catheter system may include a catheter shaft having a lumen defined therein and an outer wall surface having an expandable medical balloon and a sheath disposed thereon. A first guidewire may be disposed in the lumen of a needle, and a second guidewire may be disposed in the lumen of the needle. The method may also include advancing a needle into the desired organ, advancing a guidewire through the needle, advancing a catheter system having an expandable medical balloon and a sheath over the guidewire through the needle lumen, and performing removal of an obstruction in the organ through the sheath of the catheter system.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosure.

The present devices and methods can be used for a variety of percutaneous procedures for removing objects from a body organ by inserting a needle through an incision in the skin into the body organ. For example, a guidewire may be advanced through the needle into the body organ and positioned adjacent the object. A catheter device including a balloon and/or a preloaded sheath may then be advanced over the guidewire until the distal tip of the balloon is in the organ. The balloon is expanded and the catheter withdrawn leaving the sheath in place. These procedures may involve one or more catheter devices. Removal of the objected is performed through the sheath, often by first aspirating a contrast media or other means of visual detection into the body organ.

In one example minimally invasive procedure, percutaneous nephrolithotomy (PCNL) is used to remove stones from the kidney. For example, a small incision of about 1 cm or less may be made in the skin of a patients' back on one side of the spine or the other. A needle is then passed through the incision into the pelvis of the kidney into a selected calyx and aspiration of contrast media can be used to confirm the position. A guidewire may be passed through the needle into the pelvis. The needle is then withdrawn with the guide wire still inside the pelvis. Dilatation balloons can then be passed over the guidewire followed by introduction of a working sheath. Introduction of the working sheath maybe accomplished through the use of a catheter that has been preloaded with a sheath. The catheter is removed once the sheath is in place. Medical procedures can then be performed by the surgeon through the sheath. This can include passing a nephroscope through the sheath, and removing small stones. In case the stone is larger, it may first have to be crushed using ultrasound probes and then the stone fragments are removed.

The procedure may include insertion of a guidewire followed by an occlusion balloon which is inflated at the desired position, as well as other intermediate procedures including placement of a wire or the like. The procedure is suitably used to remove stones (e.g., relatively “large” stones that may be more about 2 cm in size or more) and which are present near the pelvic region. It is usually done under general anesthesia or spinal anesthesia.

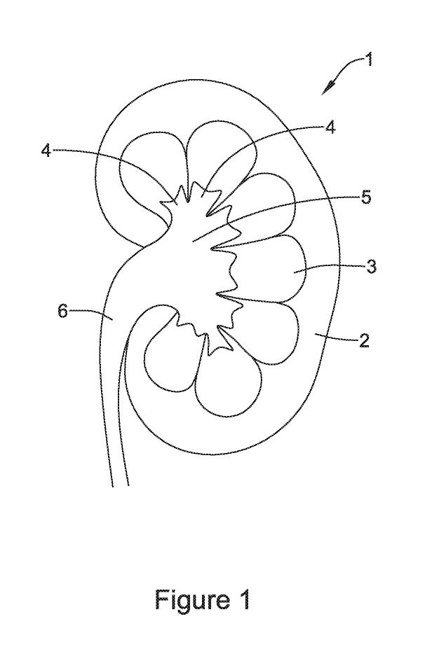

Turning now to the figures, FIG. 1 is a schematic diagram illustrating an overview of a kidney 1. The kidney 1 includes a renal cortex 2, typically 5 calices, each calyx 4 having medulla 3, a renal pelvis 5, and a ureter 6 connected thereto.

FIG. 2 is a schematic diagram illustrating a portion of an example PCNL procedure for retrieval of kidney stones, wherein a needle 9 has been inserted into the patient's back through an incision in the skin. The needle 9 is inserted into the kidney 1 through the renal cortex 2, into the medulla 3 of the selected calyx 4 past a kidney stone 11, and into the renal pelvis 5. A guidewire 10 is then inserted through the needle 9 and positioned into the ureter 6. Now, the stone 11 may be removed.

It can be difficult to maneuver guidewire 10 past the stone 11 or between stone 11 and a vessel wall. Moreover, guidewire 10 can be inadvertently dislodged from the ureter 6, for example, by inadvertent bumping of the guidewire 10 by a surgeon during a medical procedure. The example guidewires and medical device systems disclosed herein provide improved maneuverability and/or anchoring of the guidewires during a medical procedure.

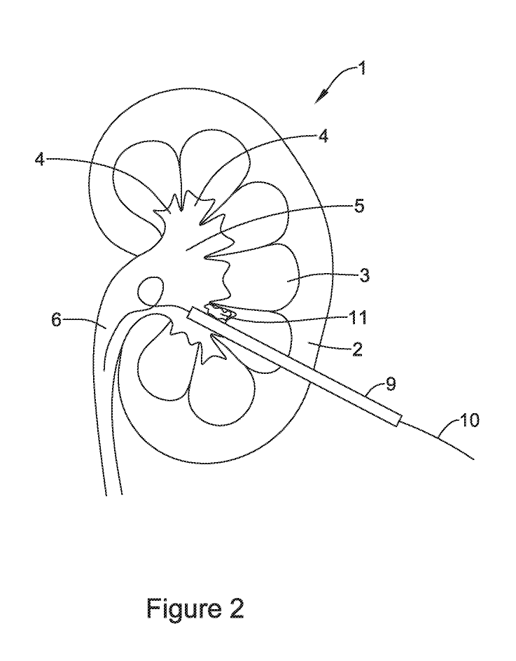

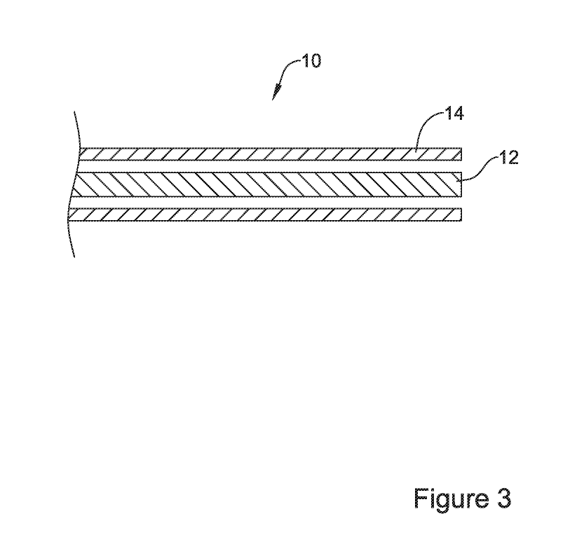

FIG. 3 is a cross-sectional view of a guidewire 10 having an inner member 12 and an outer member 14. Guidewire 10 is shown schematically. In some instances, guidewire 10 may include other structural features. In addition, inner member 12, outer member 14, or both may include one or more structural features. Inner member 12 may be movable, semi-movable or removable with respect to outer member 14. In some instances, a space or lumen is may be present between inner member 12 and outer member 14. This may allow for fluids to be passed between inner member 12 and outer member 14. In other instances, inner member 12 and outer member 14 may be relatively tightly tolerances so that little space is formed therebetween.

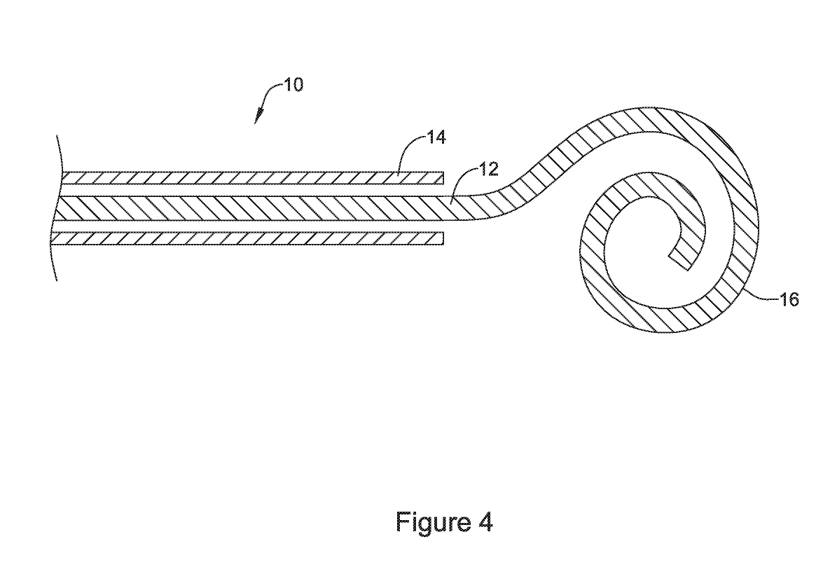

In some instance, in use, when the outer member 14 is pulled back in a proximal direction, to shift the distal end 16 of the inner member 12 into an alternative shape. In some embodiments, the distal end 16 of the inner member 12 is shifted into an expanded state to a geometric shape that is configured to anchor the guidewire with respect to its surroundings. For example, as shown in cross-section in FIG. 4, the distal end 16 of the guidewire 10, expands into a spiral configuration. Examples of such geometric shapes include, but are not limited to, cylindrical, helical, elliptical, umbrella shaped, tubular or a combination thereof.

Shifting of the distal end 16 of the inner member 12 into an alternative or expanded state may be induced by exposure to an increase in temperature, by inflating the distal end 16 of the inner member 12 into an expanded state using inflation fluid, by exposure to an electric field, or combinations thereof. For example, inner member 12 may include a shape memory material such as a nickel-titanium alloy (e.g., nitinol). Other materials are contemplated.

In some embodiments, the outer member 14 may be formed of relatively stiff material and inner member 12 may be formed from a more flexible material.

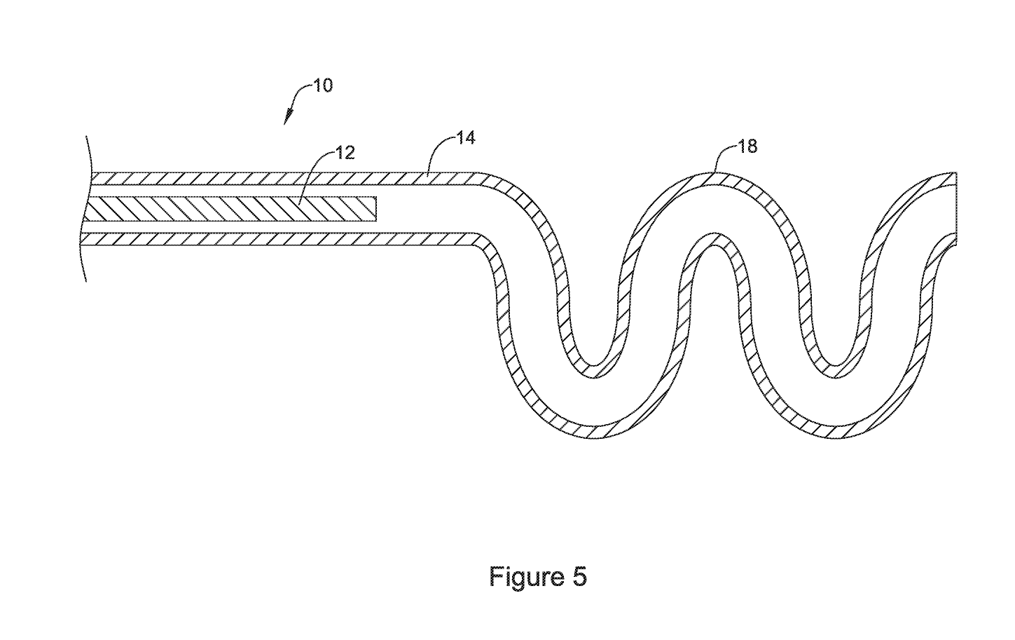

FIG. 5 is a cross-sectional view of another embodiment of a guidewire 10 having an inner member 12 and an outer member 14. In this embodiment, the outer member 14 may be formed from a relatively flexible material relative to the inner member 12 and the distal end 18 of the outer member may be configured to taken on an alternative shape as described above with respect to the distal end 16 of the inner member 12. For example, proximal retraction of inner member 12 may allow outer member 14 to shift to an alternative shape or configuration.

It is also noted the distal end 16 of the inner member 12 or the distal end 18 of the outer member 14 may be formed from a material that is different than the rest of the inner member 12 or outer member 14, and composites of materials may be employed as well.

The inner member 12 and the outer member 14 can be formed from any suitable guidewire material including metals, metal alloys, and polymer materials. The shape of the distal end 16 can be any of those disclosed herein.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In some embodiments, the more flexible of the inner member 12 and the outer member 14 may be formed from, for example, a shape memory material including shape memory polymers and metal alloys, a polymer material, or an electroactive polymer material.

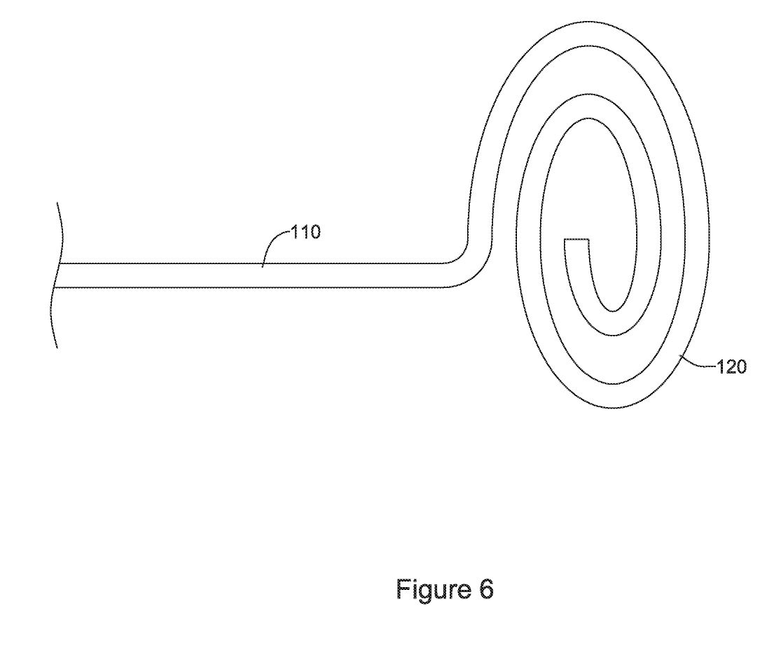

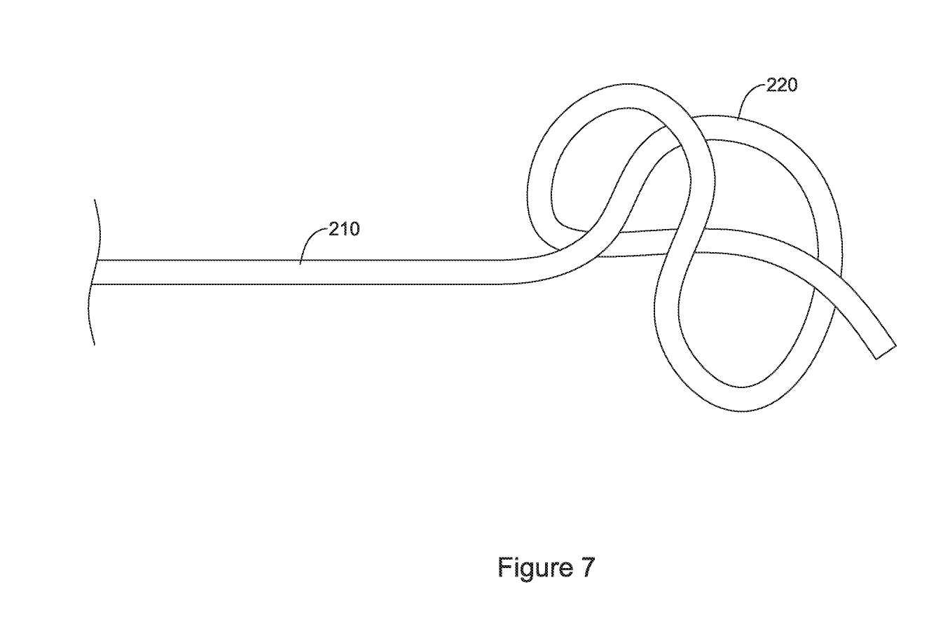

FIGS. 6 and 7 are side views illustrating alternative shapes for the distal end 120 of a guidewire 110 and the distal end 220 of a guidewire 210. As discussed above, the distal end 110 or 220 are not limited to these configurations or shapes. The distal end 120 or 220 in FIGS. 6 and 7, may be configured to shift to an alternative shape in order to anchor the guidewire in position to its surrounds, such as in a body organ.

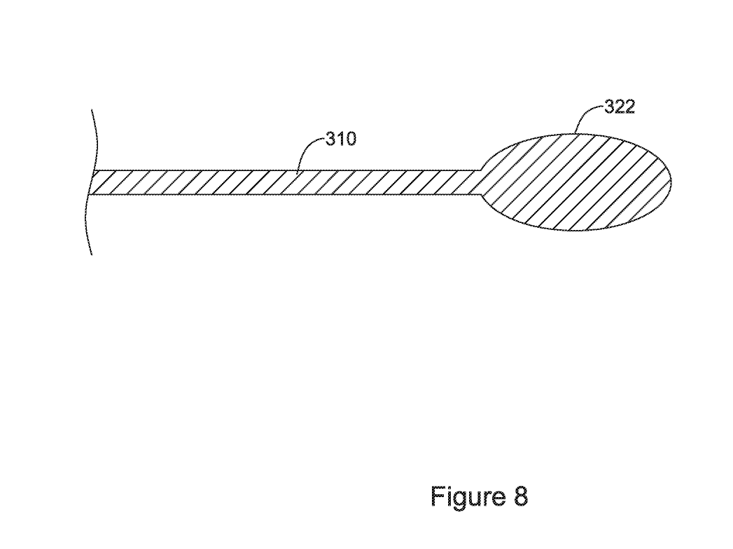

FIG. 8 is a cross-sectional view of another example embodiment of a guidewire 310 having an alternative distal end 322. In this embodiment, distal end 322 of guidewire 310 is configured to maneuver between an occlusion and a vessel wall in a patient's body. Examples of geometric shapes which may be employed include, but are not limited to, flat, spatula shaped, spoon shaped, cylindrical, elliptical, curved, or segmented with interlocking pieces. In this embodiment, the distal end 211 is spatula or spoon shaped.

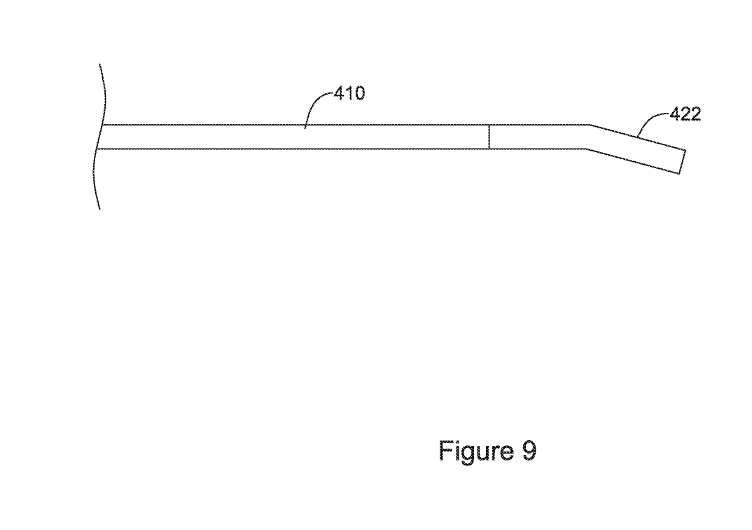

FIG. 9 is partial end view of a guidewire 410 having a distal end 422 having a bent shape configured for maneuvering around or between an occlusion and a vessel wall. This is an alternative shape. Other shapes are contemplated.

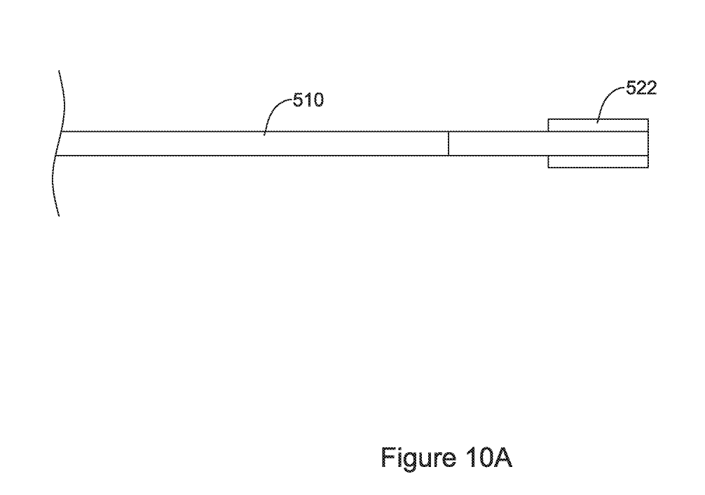

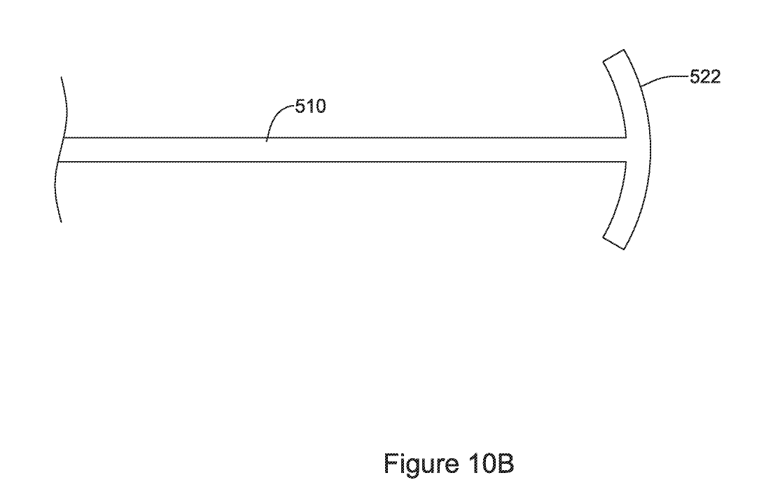

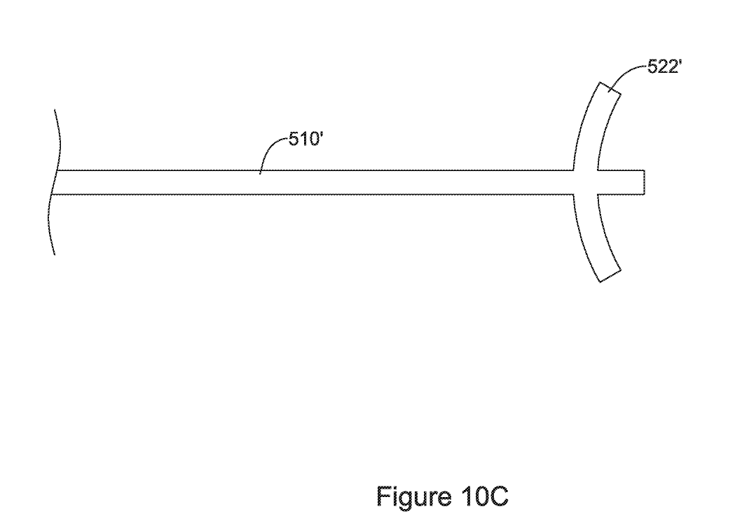

FIG. 10A is a side view of a guidewire 510 with a distal end region 522 in an unexpanded state. FIGS. 10B and 10C are side views of a guidewire 510 similar to that shown in FIG. 10A in expanded states having alternative umbrella shapes. In some instances, the distal end region 522 may be attached to the guidewire 10 at one end and free from attachment at the other end. The distal end region 522 can then shift to a second configuration.

For example, the distal end region 522 may be attached to the guidewire 10 at the distal end of the distal end region and when the guidewire 510 shifts to an expanded shape, the proximal end of the distal end region may shift radially outward as shown in FIG. 10B. Alternatively, the distal end region 522 may be attached to the guidewire 10 at the proximal end of the distal end region and when the guidewire 510 shifts to an expanded shape, the distal end of the distal end region may shift radially outward as shown in FIG. 10C.

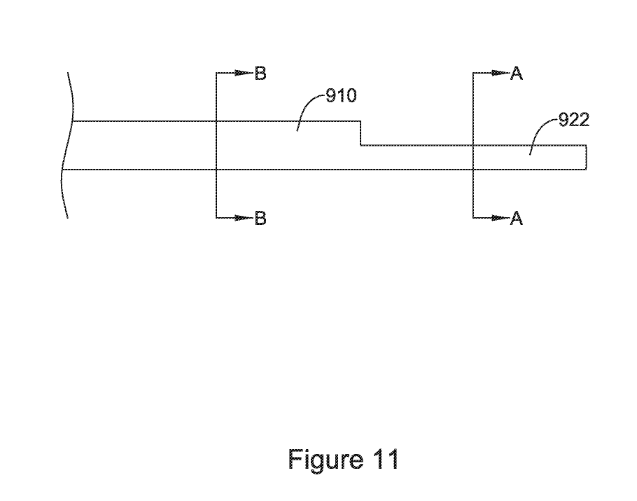

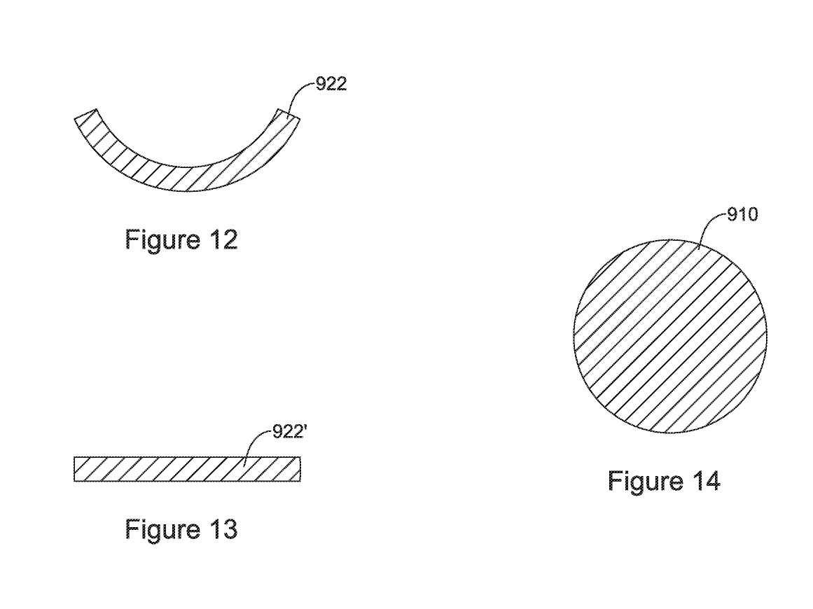

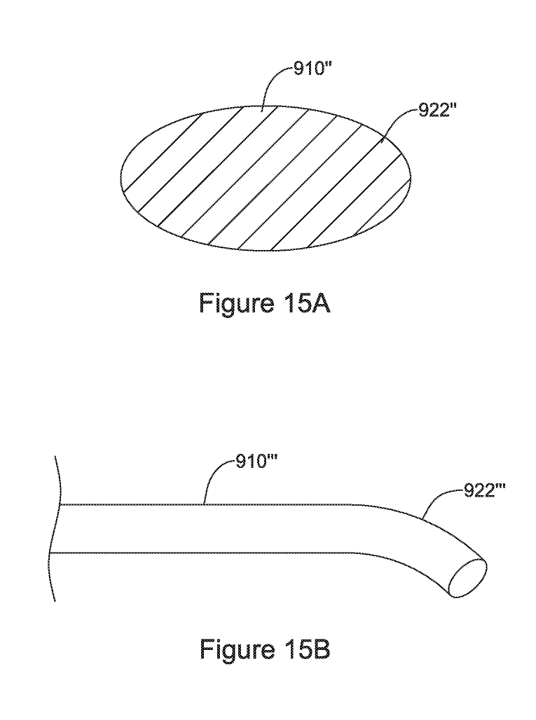

FIGS. 11-15B illustrate some of the other shapes contemplated for various guidewires disclosed herein. For example, FIG. 11 is a side view of a guidewire 910 having a distal end 922 with another alternative shape. FIG. 12 is cross-sectional view a guidewire 910 wherein the distal end 922 is curved. FIG. 13 is a cross-sectional view of the guidewire 910 wherein the distal end 922′ is flat. FIG. 14 is a cross-sectional of the guidewire 910 wherein the distal end 922″ of the guidewire 910 is cylindrical or round. FIG. 15A is an end view of a guidewire 910″ wherein the distal end 922″ of the guidewire 910″ is oval or the guidewire 910″ and the distal tip 922″ are oval. FIGS. 12-15A can be taken at section A-A and/or section B-B of FIG. 11, as well as mixing and matching of the alternative shapes. FIG. 15B is a side view of an alternative embodiment of a guidewire 910″′ has a distal curved end 922″′ and may be combined with the embodiments disclosed with respect to FIG. 15A. Other guidewires are contemplated that utilize a curved distal end similar to the curved distal end 922″′.

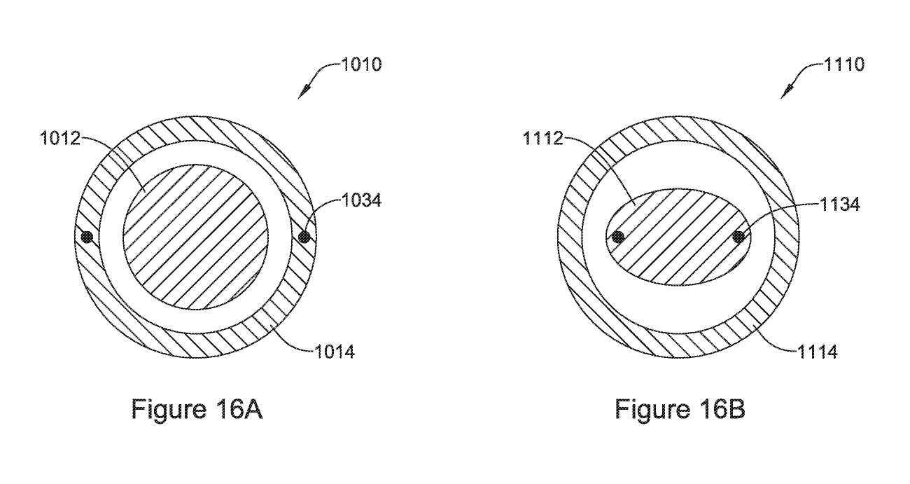

FIG. 16A is a partial cross-sectional view of a guidewire 1010 having an inner member 1012 and an outer member 1014. In this embodiment, outer member 1014 has two wires 1034 disposed therein. The wires 1034 may be manipulated at the distal end of the guidewire 1010 to bend the distal end of the guidewire 1010 to maneuver between an occlusion and a vessel wall.

FIG. 16B is a partial cross-sectional view of a guidewire 1110 having an inner member 1112 and an outer member 1114. In this embodiment, inner member 1112 has two wires 1134 disposed therein. The wires 1134 may be manipulated at the distal end of the guidewire 1110 to bend the distal end of the guidewire 1110 to maneuver between an occlusion and a vessel wall.

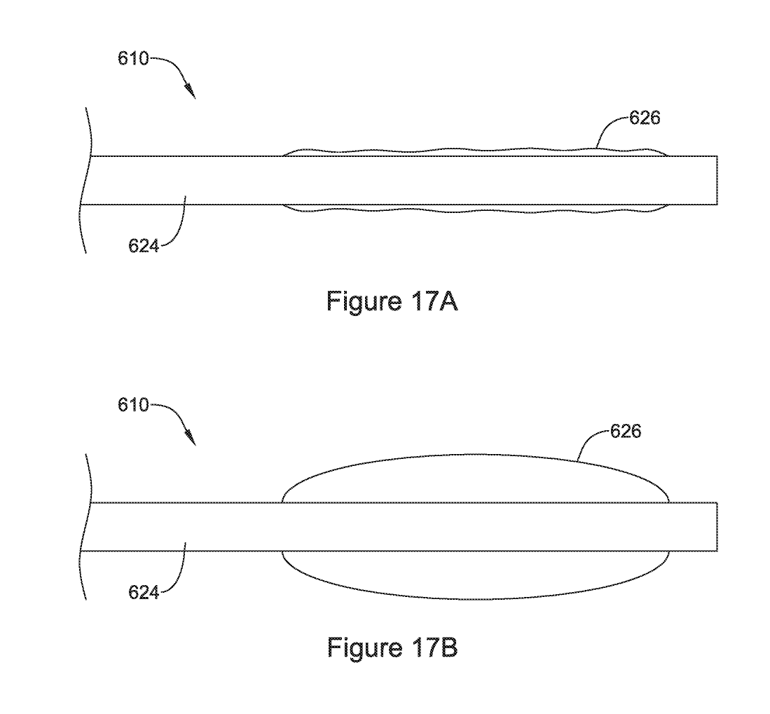

FIG. 17A is a side view of a guidewire 610 having a distal end 624 with an expandable member 626 shown in an unexpanded state. FIG. 17B is a side view of a guidewire 610 similar to that shown in FIG. 17A having a distal end 624 with an expandable member 626 shown in an expanded state. The guidewire 610 can be deliverable to the treatment site when the expandable member 626 is in an unexpanded or deflated state as shown in FIG. 17A. Once at the treatment site and the distal end of the guidewire 610 is in the targeted organ, for example, the ureter, the expandable member 626 can be expanded using an inflation fluid, for example, as shown in FIG. 17B to anchor the guidewire into position. Once the treatment is complete, the expandable member 626 can be deflated and the guidewire 610 removed from the patient's body.

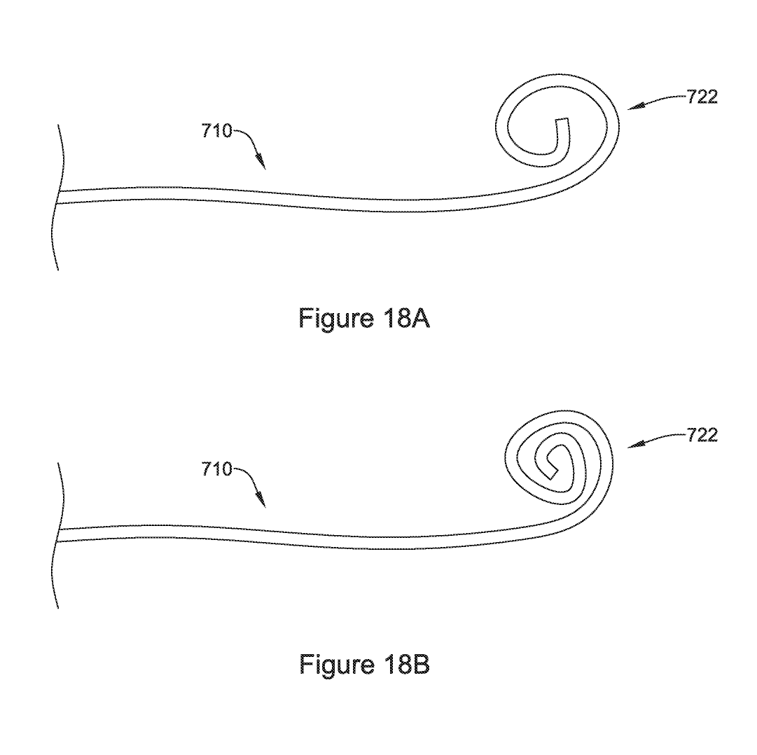

FIG. 18A is a side view of a guidewire 710 having a distal end 722 that coils upon deployment. FIG. 18B is a partial cross-sectional view of a guidewire 710 similar to that shown in FIG. 18A when tension is applied at the proximal end of guidewire 710. This embodiment can be employed with other embodiments disclosed herein, for example, the embodiments disclosed with respect to FIGS. 16A and 16B.

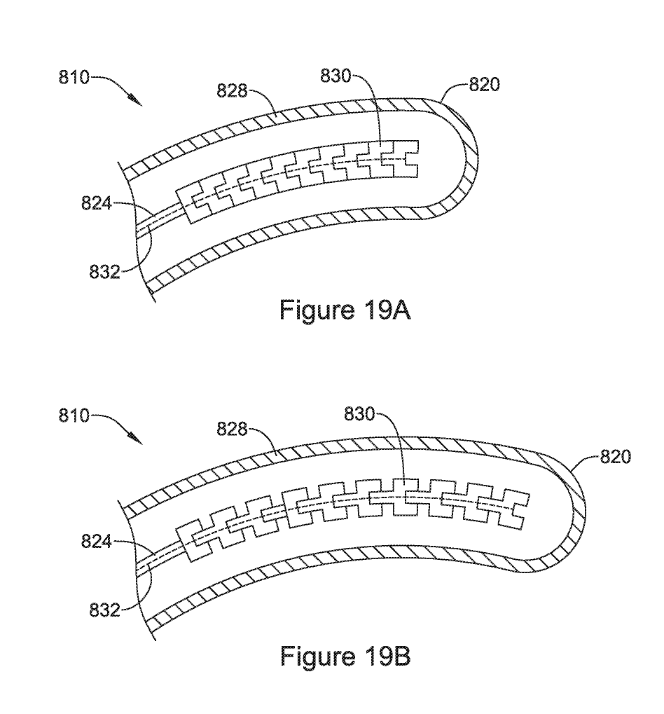

FIGS. 19A and 19B are side views of a distal end 820 of a guidewire 810 having an inner member 824 and an outer member 828. Inner member 824 includes a distal portion having interlocking segments 830 having a proximal portion having a wire 832 connected with and running therethrough. When tension is applied to the proximal end of the inner member 824 of guidewire 810, the interlocking segments 830 move with respect to the outer member 828, nest together and lock into place as shown in FIG. 19A, and when the proximal end of the inner member 824 of the guidewire 910 is pushed from the proximal the interlocking segments spread apart as shown in FIG. 19B allowing the distal end 1220 of the guidewire 810 to become more flexible for maneuvering around an occlusion.

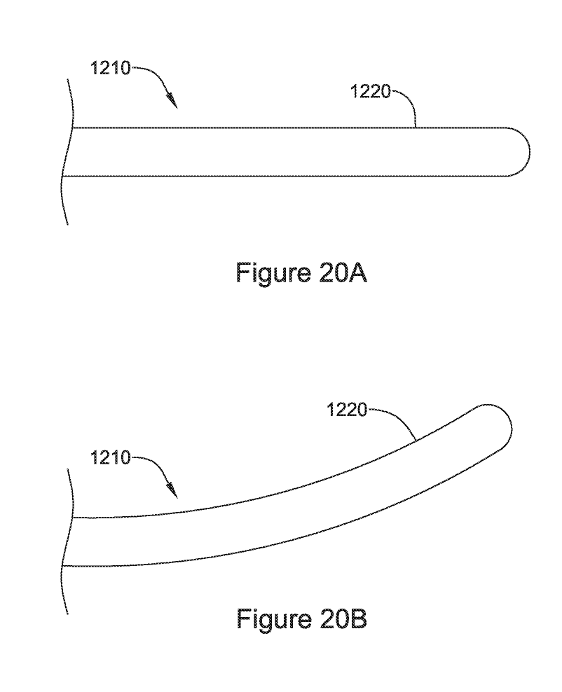

FIGS. 20A is a side view of a distal end 1220 of a guidewire 1210 that is shiftable into an alternative shape upon activation such as electrical current. FIG. 20B is an end view of the distal end 1220 of a guidewire 1210 similar to that shown in FIG. 20A that has been shifted into a curved distal end 1220. One example of a suitable material useful for forming at least the distal end 1220 of guidewire 1210 in this embodiment is an electroactive polymer material.

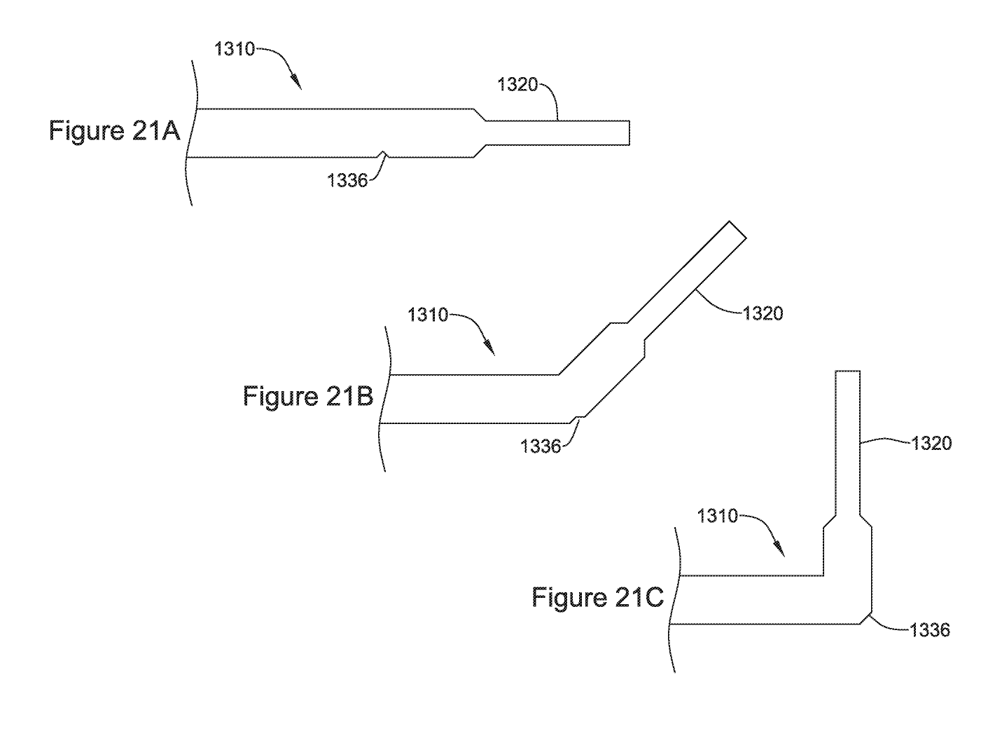

FIGS. 21A-21C are side views of an alternative embodiment of a guidewire 1310 that is configured to bend at notch 1336. Guidewire 1310 is further shown with a flat tip 1320 for maneuvering between an occlusion and a vessel wall. When the proximal end of the guidewire 1310 is pushed in a distal direction, the guidewire 1310 bends partially and notch 1336 as shown in FIG. 21B and when further pushed in a distal direction, it bend at a right angle as shown in FIG. 21C. This allows a surgeon to maneuver the guidewire past an occlusion in a body lumen without steering controls.

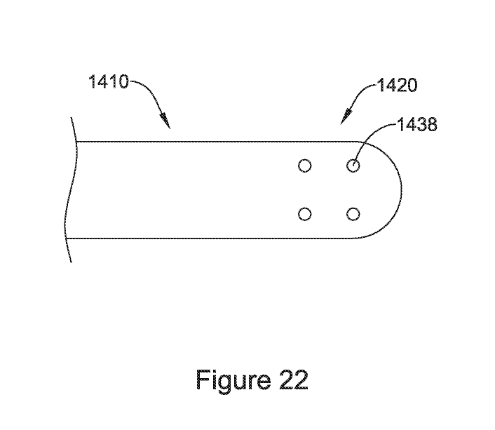

FIG. 22 is a side view of an alternative embodiment of a guidewire 1410. In this embodiment guidewire 1410 is a hollow member 1420 having openings 1438 extending through the wall of the guidewire. Contrast fluid may be forced through openings 1438 for visualization of an occlusion using fluoroscopy to maneuver the guidewire past an occlusion in a body lumen.

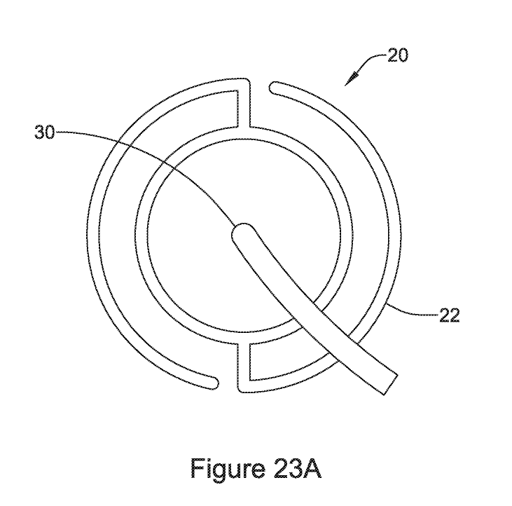

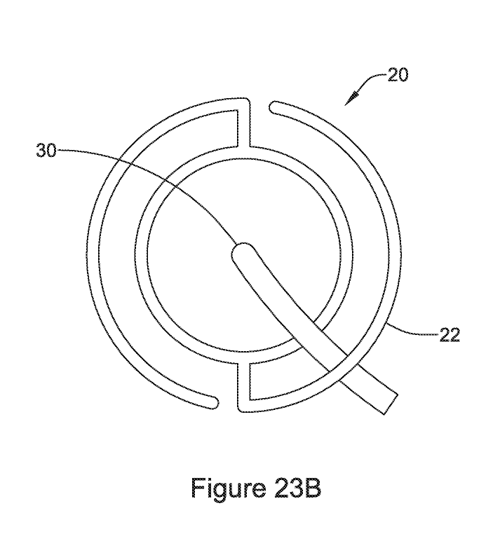

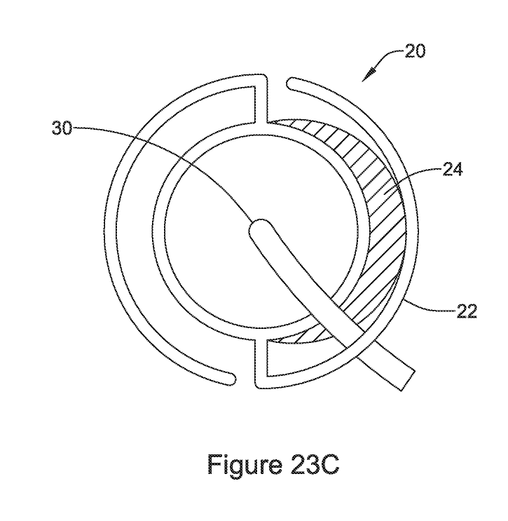

FIG. 23A is an end view of a proximal end of a sheath 20 having a guidewire locking device 22 with a guidewire 30 disposed in sheath 20. Sheath 20 includes a locking device in the form of wings 22, for example with an opening therein to maneuver the proximal end of the guidewire 30 into a wing 22 for locking the proximal end of the guidewire 30 in place to prevent bumping of the proximal end of the guidewire 30 which can dislodge the distal end of the guidewire 30 from a body lumen during a medical procedure.

FIG. 23B is an end view of the proximal end of the sheath with the proximal end of the guidewire 30 extending from the sheath 20 and dispose within wing 22 to lock the guidewire 30 in place.

FIG. 23C is an end view of a proximal end of a sheath 20 with the proximal end of the guidewire 30 extending from the sheath 20 and disposed within wing 22 locking the guidewire in place. In this embodiment, sheath 20 further includes an inflatable member 24 in wing 22 which upon inflation, which can provide more secure locking of the guidewire 30 in between inflatable member 24 and wing 22.

1. A guidewire, comprising:
an elongate inner member having a proximal portion and a distal portion;
an elongate outer member disposed about the elongate inner member; and
wherein one of the elongate inner member and the elongate outer member is more flexible relative to the other of the elongate inner member or the elongate outer member, and the elongate inner member is movable relative to the elongate outer member.

2. The guidewire of claim 1 wherein the elongate outer member is movable with respect to the elongate inner member, the elongate outer member can be pulled in a proximal direction relative to the elongate inner member, to shift the distal portion of the elongate inner member into an alternative shape.

3. The guidewire of claim 1 wherein the elongate inner member is movable with respect to the elongate outer member, the elongate inner member can be pulled in a proximal direction relative to the elongate outer member, to shift the distal portion of the elongate outer member into an alternative shape.

4. The guidewire of claim 1 wherein the distal portion of the elongate inner member is shiftable into an expanded state, in the expanded state, the distal portion of the elongate inner member comprises a geometry configured to anchor the guidewire with respect to its surroundings.

5. The guidewire of claim 4 wherein the distal portion of the elongate inner member is shifted into the expanded state by exposure to an increase in temperature.

6. The guidewire of claim 4 wherein the distal portion of the elongate inner is shifted into an expanded state by inflating the distal portion with an inflation fluid.

7. The guidewire of claim 4 wherein the distal portion of the elongate inner member is shifted into an expanded state by exposure to an electric field.

8. The guidewire of claim 4 wherein at least the distal portion of the elongate inner member comprises a polymer material, a shape memory material, an electroactive polymer material or combinations thereof.

9. The guidewire of claim 8 wherein the elongate inner member comprises a shape memory material, the shape member material is a nickel-titanium alloy.

10. The guidewire of claim 4 wherein the geometry of the distal portion of the elongate inner member in the expanded state is cylindrical, helical, elliptical, umbrella shaped, tubular, or a combination thereof.

11. The guidewire of claim 1 wherein the distal portion of the elongate outer member is shiftable into an expanded state providing a geometry configured to position the guidewire with respect to its surroundings, the distal portion of the elongate outer member is shifted into the expanded state by an increase in temperature, by inflating with an inflation fluid or by exposure to an electric field.

12. The guidewire of claim 1 wherein the elongate inner member has a distal tip, the distal tip has a shape including flat, spatula shaped, spoon shaped, cylindrical, elliptical, curved, segmented with interlocking pieces, or a combination thereof.

13. The guidewire of claim 12 wherein the elongate inner member comprises stainless steel, nickel-chromium alloy, cobalt alloy, nickel-titanium alloy, or combinations thereof.

14. The guidewire of claim 12 wherein at least the distal portion of the elongate inner member comprises a shapeable material.

15. A guidewire, comprising: a shaft, the shaft having a distal tip that is flat, spatula shaped, spoon shaped, cylindrical, elliptical, curved, or segmented with interlocking pieces, the distal tip is configured to navigate past an occlusion in a body lumen.

16. A method of retrieving an object from a body organ percutaneously, the method comprising:
inserting a needle into the body organ;
advancing a guidewire through the needle into the body organ to position adjacent the object, the guidewire comprising an elongate inner member and an elongate outer member is disposed about an outer surface of the elongate inner member, one of the elongate inner member or elongate outer member is more flexible that the other of the elongate inner member and the elongate outer member, the elongate outer member is movable with respect to the elongate inner member;
pulling back the elongate outer member or the elongate inner member to release a distal portion elongate outer member or the elongate inner member;
advancing a catheter comprising an expandable medical balloon having a distal tip over the guidewire until the distal tip of the balloon is in the organ;
expanding the medical balloon;
providing a sheath over the catheter; and
performing removal of the object from a body organ through the sheath.

17. The method of claim 16 wherein one of the elongate outer member or the elongate inner member has a distal tip expandable to an anchor shape, wherein pulling back one of the elongate outer member or the elongate inner member anchors the guidewire in the organ.

18. The method of claim 17 wherein the anchor shape comprises a geometry including cylindrical, helical, elliptical, umbrella shaped, tubular, or a combination thereof.

19. The method of claim 16 wherein the elongate inner member has a distal tip that is configured for maneuverability past an occlusion in a body lumen, the distal tip is flat, spatula shaped, spoon shaped, cylindrical, elliptical, curved, segmented with interlocking pieces, or a combination thereof.

20. The method of claim 16, wherein the method is percutaneous nephrolithotomy, the body organ is a kidney, and the object is a kidney stone.
